(19) United States
(12) Patent Application Publication
(10) Pub. No.: US 2020/0188625 A1
(43) Pub. Date: Jun. 18, 2020

(54) COOLING DEVICE AND PROCESS FOR COOLING FOR A CLOSED-CIRCUIT RESPIRATOR

(71) Applicant: Dräger Safety AG & Co. KGaA, Lubeck (DE)

(72) Inventor: Henning GERDER, Lubeck (DE)

(21) Appl. No.: 16/716,985

(22) Filed: Dec. 17, 2019

(30) Foreign Application Priority Data
Dec. 18, 2018 (DE) 10 2018 009 803.7

(51) Int. Cl. A61M 16/10; A61M 16/00

(52) U.S. Cl. CPC A61M 16/1075; A61M 16/0093; A61M 2205/3606; A61M 2205/60; A61M 2205/127; A61M 2205/3368; A61M 2202/0225

(57) ABSTRACT

A cooling device for a closed-circuit respirator includes a device housing and a coolant arrangement. The device housing has a gas inlet, which is configured to admit a gas to be cooled into the device housing, and has a gas outlet, which is configured to let the gas admitted through the gas inlet into the device housing out of the device housing. The coolant arrangement is arranged in the device housing and the coolant arrangement has a first coolant with a first melting point T1 and a second coolant with a second melting point T2. The first coolant and the second coolant are arranged in the coolant arrangement such that no direct contact is possible between the first or second coolant and the gas being cooled. The first melting point T1 is different from the second melting point T2.

### CROSS REFERENCE TO RELATED APPLICATIONS

This application claims the benefit of priority under 35 U.S.C. § 119 of German Application 10 2018 009 803. 7, filed Dec. 18, 2018, the entire contents of which are incorporated herein by reference.

### TECHNICAL FIELD

The present invention pertains to a cooling device for a closed-circuit respirator, to a closed-circuit respirator and to a process for operating a closed-circuit respirator.

### TECHNICAL BACKGROUND

The use of a cooling device in a closed-circuit respirator for cooling a breathing gas stream is known and necessary. Thus, a lime material, which is typically used as an absorber for treating the gas and which treats the breathing gas by removing CO₂, produces heat continuously. In the closed breathing circuit, this causes, over the duration of use of the closed-circuit respirator, the temperature of the inhaled gas to rise for the user of the closed-circuit respirator into a temperature range that is at least extremely uncomfortable for the user during the inhalation. Provisions are therefore made for a continuous cooling of the breathing gas circuit by a cooling device. The cooling device has a coolant, which is typically cooled to below its melting point prior to the use of the closed-circuit respirator.

The coolant is preferably ice or a coolant, which is configured as a phase-change material (PCM) and is used within a liquid container/heat exchanger in the closed-circuit respirator.

### SUMMARY

An object of the present invention is to provide an improved cooling for a closed-circuit respirator, especially a cooling with improved controllability of a breathing gas temperature curve in a closed-circuit respirator.

To accomplish this object, a cooling device with a device housing and with a coolant arrangement is proposed according to the present invention for a closed-circuit respirator.

The device housing is configured to be able to be arranged within a breathing gas circuit in the closed-circuit respirator, and the device housing has, furthermore, a gas inlet, which is configured to admit a gas to be cooled into the device housing, and a gas outlet, which is configured to let the gas admitted into the device housing flow out of the device housing. The device housing gas, furthermore, a device volume, which is enclosed by a housing wall of the device housing, the device housing being configured such that the gas to be cooled can flow from the gas inlet through the device volume to the gas outlet. The gas to be cooled is preferably guided from the gas inlet through the device volume to the gas outlet.

The coolant arrangement is arranged in the device volume and the coolant arrangement has a first coolant with a first melting point T1 and a second coolant with a second melting point T2, the first coolant and the second coolant being arranged in the coolant arrangement such that no direct contact is possible between the first or second coolant and gas to be cooled. The first melting point T1 is different from the second melting point T2.

The present invention is based on the discovery that even though a quantity of oxygen being carried along in a pressurized cylinder, a dimensioning of a quantity of carried-along lime in a CO₂ absorber and a cooling capacity are adapted to one another for the use of closed-circuit respirators, standardized ambient temperatures and standardized service lives are typically assumed. It was recognized that peculiarities, such as high outside temperatures and very short or very long durations of use, require the ability to set a temperature curve of the coolant used in the closed-circuit respirator. This problem is addressed according to the present invention by the use of at least two different coolants with different melting points T1 and T2.

The solution according to the present invention makes it advantageously possible to combine the cooling effects of two different coolants for use in the cooling device of a closed-circuit respirator. An especially long service life of the closed-circuit respirator, especially a service life longer than 240 minutes, is made possible thereby with an inhaled gas temperature below 42°.

The effect of a corresponding enthalpy of fusion, which is removed from the gas being cooled for the melting of the corresponding coolant, starts at different times of use of the closed-circuit respirator. As a result, a desired temperature curve of the inhaled gas over time can be obtained, especially with a corresponding, additional cooling due to the removal of the enthalpy of fusion, when the corresponding melting point is present.

For especially short-term uses or for use at very high outside temperatures, which are possible, for example, during uses in firefighting, the cooling device according to the present invention makes possible such an advantageous configuration of the first and second coolants, which are maintained over a long time period at an especially low temperature of the coolant arrangement within the cooling device.

Furthermore, the cooling device according to the present invention has the advantage that the same cooling device can be used for different use scenarios, especially different durations of use and outside temperatures, because only the type and the composition of the first and second coolants must be adapted to the particular use scenario.

It is typically ensured by means of the housing enclosing the particular coolant that no direct contact is possible between the first coolant or second coolant and the gas to be cooled. A combination of the first and second coolants may also be present within a housing.

Preferred embodiments of the cooling device according to the present invention will be described below.

In one embodiment, the cooling arrangement comprises at least one additional coolant with another melting point, which is different from T1 and T2. As a result, the cooling device can be adapted even more precisely to a duration of use to be expected and/or to an outside temperature to be expected during the use of the closed-circuit respirator. In an alternative embodiment, the coolant arrangement comprises precisely the first coolant and the second coolant. This makes possible an especially simple and rapid performance of the filling of the coolant arrangement with coolant, which is especially advantageous under the conditions of use.

In an especially preferred embodiment, the first melting point T1 and the second melting point T2 are each lower than 50° C., especially lower than 45° C., preferably lower than 40° C., and they are especially preferably between 30° C. and 35° C. In this embodiment, the cooling device makes it possible for the temperature of the inhaled gas provided to be lower than 50° C., especially lower than 44° C., and preferably lower than 40° C. over a predefined time period. Such a reduced temperature of the inhaled gas increases the comfort for a user of the closed-circuit respirator. In particular, it is desirable for the temperature of the inhaled gas provided to be lower than 42° C. The particular melting point of the coolant is relevant for the control of the cooling provided due to the fact that a nearly linear rise in the temperature of the provided inhaled gas over time can be expected until the melting point is reached, whereas a reduction of the temperature of the inhaled gas can be expected to start over several minutes, especially over more than 30 minutes, in the range of the melting point based on the enthalpy of fusion needed for the melting before a nearly linear rise will then take place. A close examination of this curve is discussed in connection with FIG. 4.

In an especially preferred embodiment, the coolant arrangement is configured such that the gas to be cooled is sent from the gas inlet through the coolant arrangement to the gas outlet. The gas to be cooled is preferably sent in this case through at least one first area containing the first coolant and through at least one second area containing the second coolant. Such a guiding of the gas is advantageous especially due to the fact that blocking of a gas flow between the gas inlet and the gas outlet is avoided. As a result, a constant inhalation resistance, which is felt to be pleasant, is provided for a user of the closed-circuit respirator. Furthermore, this embodiment has the advantage that cooling is ensured by the first coolant and by the second coolant. At least major inhomogeneities of the temperature of the gas being cooled are avoided thereby.

In an advantageous embodiment of the cooling device according to the present invention, the first coolant and/or the second coolant are formed by a phase-change material (PCM) as a cooling material consisting of paraffin or a salt material. The use of a PCM material makes possible the advantageous composition of this material in the solid state already at room temperature. Furthermore, these materials with different melting points are commercially available. Among other things, the use of PCM31, which has a melting point of 31° C., and the use of PCM37, which has a melting point of 37° C., are known. A coolant is formed by water in an additional or alternative embodiment.

In one embodiment according to the present invention, the first coolant and/or the second coolant are provided by a plurality of individually sealed capsules filled with the respective coolant. Such capsules can be introduced into the device housing as a bulk material in an especially simple manner. An additional air volume within the capsule must be taken into consideration when filling the capsules with the respective coolant in order to make possible an expansion of the coolant above the melting point. The use of capsules makes it possible, in particular, to obtain a certain ratio of the first coolant to the second coolant within the cooling arrangement in an especially simple manner. In one variant of this embodiment, a particular capsule of a plurality of capsules has at least one of the following shapes: Hemispherical shape, frustoconical shape, conical shape, pyramid shape, truncated pyramid shape, and tetrapod shape. Such a shape for the capsules is advantageous, because it is possible to guide the gas to be cooled through the coolant arrangement due to the cavities formed as a result. In a preferred variant of this embodiment, the coolant arrangement has at least one capsule rack, which is configured to accommodate a plurality of capsules. The capsule rack is preferably arranged in this case such that the gas to be cooled is guided between the gas inlet and the gas outlet along the capsule rack or through the capsule rack.

In an embodiment that is additional or alternative to the previous embodiment, the first coolant and/or second coolant are provided by a plurality of individually sealed heat exchanger plates filled with a respective coolant. The use of heat exchanger plates according to this embodiment makes it possible in an especially advantageous manner to guide the gas to be cooled between the gas inlet and the gas outlet through a corresponding array of heat exchanger plates. Furthermore, the use of heat exchanger plates makes possible an especially simple storage of the heat exchanger plates between the uses of the closed-circuit respirator. An air volume must be provided within the heat exchanger plate in order to make possible an expansion of the coolant above the melting point. In an especially preferred variant of the above embodiment, the coolant arrangement is formed by an alternating sequence of heat exchanger plates filled with the first coolant and of heat exchanger plates filled with the second coolant. According to this variant, a contact is advantageously provided for the gas to be cooled with heat exchanger plates filled with the first coolant and with heat exchanger plates filled with the second coolant. According to this variant, a contact of the gas to be cooled with heat exchanger plates filled with the first coolant and with heat exchanger plates filled with the second coolant is made possible. Such a sequence of heat exchanger plates may especially advantageously form guide ducts for the gas to be cooled. A preferred example of this variant comprises such an alternating sequence that a heat exchanger plate filled with the first coolant is followed directly by a heat exchanger plate filled with the second coolant and vice versa. In another alternative or additional example of this variant, a distributing device, which is configured to guide the gas to be cooled from the gas inlet in guide ducts formed by a plurality of heat exchanger plates, is formed in the area of the gas inlet. The heat exchanger plates are advantageously located at differently spaced locations from one another corresponding to a gas pressure within the distribution device.

The device housing preferably has a plurality of plate-mounting tracks for inserting the heat exchanger plates, a respective pair of plate-mounting tracks being arranged at opposite housing walls of the device housing in order to arrange a heat exchanger plate within the device volume. An especially simple manual insertion of the respective heat exchanger plate in a predefined position is made possible thereby.

In another embodiment, the cooling device according to the present invention has, furthermore, a temperature sensor unit, especially a temperature sensor unit embodied with an radio frequency identification (RFID) tag, which is arranged at or on the device housing and which is configured to output a signal of a temperature currently present in the device volume in case of a corresponding external temperature polling. The configuration of an RFID tag suitable for the temperature measurement is known from U.S. Pat. Nos. 6,563,417 B1, 6,847,912 B2 and 6,953,919 B2 (6,563,417 B1, 6,847,912 B2 and 6,953,919 B2 are each incorporated herein by reference). This is an example of RFID technology with a temperature sensor as provided with this embodiment. Whether a cooling of the cooling device, set on the basis of the coolant composition used, is taking place as planned can advantageously be checked thereby. Such a checking is possibly due to the temperature sensor even during the use of the closed-circuit respirator. This makes it possible to individually adapt the planned duration of use during the use. The external temperature polling is preferably effected by a temperature-reading unit arranged within the closed-circuit respirator, especially by a temperature-reading unit embodied by means of an RFID reading unit.

Furthermore, a closed-circuit respirator, which has a cooling device according to at least one of the above embodiments, is provided for accomplishing the object according to the present invention.

The closed-circuit respirator according to the present invention comprises the cooling device according to the present invention and thus has all the advantages of the cooling device according to the present invention.

In a preferred embodiment of the closed-circuit respirator according to the present invention, the closed-circuit respirator has, furthermore, a temperature-reading unit, especially a temperature-reading unit embodied by means of an RFID reading unit, which is configured to output an external temperature polling and thereby to trigger an output of the temperature currently present in the device housing by a temperature sensor arranged in the cooling device, especially by a temperature sensor embodied with an RFID tag. This advantageously also makes it possible to check the temperature currently present in the cooling device during a use of the closed-circuit respirator.

According to another aspect of the present invention, the object according to the present invention is accomplished by a process for operating a closed-circuit respirator, especially for cooling a breathing gas circuit led through the closed-circuit respirator, which has the following steps:
provision of a device housing, which has a housing wall, which encloses a device volume;
provision of a first coolant with a first melting point T1 and of a second coolant with a second melting point T2 in the device volume, the first melting point T1 being different from the second melting point T2;
admission of a gas to be cooled into the device housing;
cooling of the gas to be cooled by the first coolant and by the second coolant, while no direct contact is possible between the gas to be cooled and the first or second coolant; and
letting the gas having passed through the device housing out of the device housing.

The process according to the other aspect of the present invention is preferably carried out by the cooling device according to the present invention and by the closed-circuit respirator according to the present invention. The process according to the present invention consequently has the same advantages as the cooling device according to the present invention.

In a preferred embodiment of the process according to the present invention, the first melting point T1 and the second melting point T2 are each lower than 50° C., especially lower than 45° C. and preferably lower then 40° C.

In another embodiment of the process according to the present invention, the cooling of the gas to be cooled has the following two steps:
guiding of the gas to be cooled through at least one first area of the device volume containing the first coolant, and
guiding of the gas to be cooled through at least one second area of the device volume containing the second coolant.

The guiding of the gas to be cooled corresponding to this embodiment makes advantageously possible the contact of this gas with the at least one first area and with the at least one second area, so that a nearly homogeneous temperature of the gas is possible at the gas outlet. Furthermore, the guiding of the gas to be cooled makes possible an especially efficient configuration of the cooling device, especially an especially small volume of the cooling device, because the gas is guided at least partially along a predefined path.

According to another embodiment according to the present invention, the process further includes an arranging of heat exchanger plates in the device volume, the heat exchanger plates being sealed individually and filled each with the first coolant and/or with the second coolant. The plurality of heat exchanger plates preferably form a guide for guiding the gas to be cooled along between two respective heart exchanger plates. An especially efficient cooling of the gas to be cooled is possible as a result, so that the gas must flow over an especially short path within the device housing to achieve a desired cooling.

In an especially preferred embodiment of the process, the first coolant and the second coolant are provided at a predefined ratio, the predefined ratio being set by taking an expected duration of use of the closed-circuit respirator into consideration. The process according to this embodiment makes especially advantageously use of the possibility of controlling the cooling of the breathing gas within the closed-circuit respirator by setting a predefined ratio of the first coolant to the second coolant. The course of the cooling can now be controlled over the duration of use, especially in order to make possible an especially long use of the closed-circuit respirator. As an alternative, an especially short duration of use can also be taken into consideration by the inhaled gas being cooled only briefly, but to a very low temperature, so that an especially comfortable use is possible for the user of the closed-circuit respirator.

According to another, especially preferred embodiment of the process, the first coolant and the second coolant are provided at a predefined ratio, the predefined ratio being set by taking an expected outside temperature of a use of the closed-circuit respirator into consideration. The cooling of the breathing gas can be controlled in this embodiment in an especially advantageous manner by setting the predefined ratio of the first coolant to the second coolant. In particular, a curve describing the cooling over time can be controlled. An especially intensely cooled inhaled gas can thus be provided by the closed-circuit respirator at least at the beginning of a use in case of an especially high expected outside temperature, as it may be present, for example, during a firefighting operation. As an alternative, an only slightly cooled inhaled gas may also be provided for an especially low expected outside temperature, so that a markedly longer duration of use would be possible.

In another embodiment according to the present invention, the process further includes an arranging of a temperature sensor, especially of a temperature sensor embodied by means of an RFID tag, at or in the device housing, as well as an outputting of a temperature currently present at or in the device housing, as well as an outputting of a temperature currently present in the device volume in the presence of a corresponding external temperature polling. The temperature currently present in the cooling device may also be checked during a use of the closed-circuit respirator in this embodiment.

The present invention shall now be explained in more detail on the basis of advantageous exemplary embodiments shown schematically in the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first exemplary embodiment of the cooling device according to the present invention;

FIG. 2 is a schematic view of a second exemplary embodiment of the cooling device according to the present invention;

FIG. 3 is a schematic view of a third exemplary embodiment of the cooling device according to the present invention;

FIG. 4 is a diagram showing a temperature curve of an inhaled gas over time for a cooling device according to the present invention; and

FIG. 5 is a flow chart of an exemplary embodiment of the process according to another aspect of the present invention.

### DESCRIPTION OF PREFERRED EMBODIMENTS

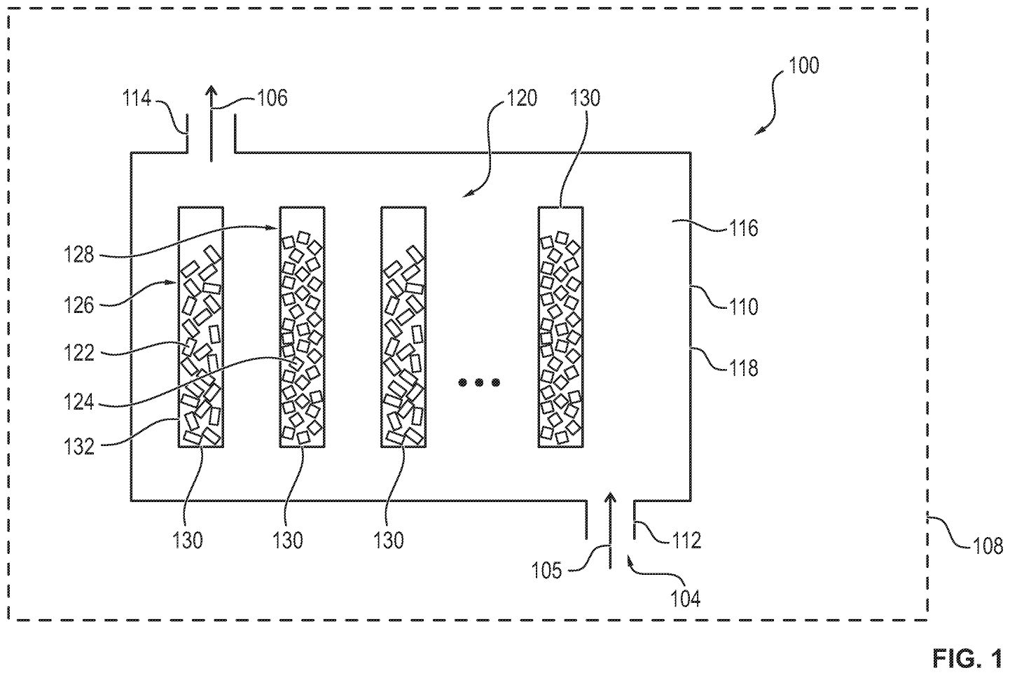

Referring to the drawings, FIG. 1 shows a schematic view of a first exemplary embodiment of the cooling device 100 according to the present invention.

The cooling device 100 comprises a device housing 110 and a coolant arrangement 120.

The device housing 110 is configured to be able to be arranged within (in fluid connection with) a breathing gas circuit 104 in the closed-circuit respirator 108. The device housing 110 of the cooling device 100 is arranged in this case within (in fluid connection with) the breathing gas circuit 104 such that the breathing gas heated by a lime used as a CO₂ absorber can be cooled again by the coolant arrangement 120. It shall be ensured hereby that an inhaled gas inhaled by the user of the closed-circuit respirator 108 will not become unpleasantly hot.

The device housing 110 has, furthermore, a gas inlet 112, which is configured to admit a gas to be cooled 105 into the device housing, and a gas outlet 114, which is configured to let the gas admitted into the device housing 110 through the gas inlet 112 flow again out of the device housing 110. Furthermore, the device housing 110 has a device volume 116, which is enclosed by a housing wall 118 of the device housing 110. The device housing 110 is configured in this case such that the gas 105 to be cooled can flow from the gas inlet 112 through the device volume 116 and to the gas outlet 114. The gas 106 passing through the gas outlet 114 is then used at least partially as inhaled gas for a user of the closed-circuit respirator.

The coolant arrangement 120 is arranged in the device volume 116 and has a first coolant 122 with a first melting point T1 and a second coolant 124 with a second melting point T2. The first coolant 122 and the second coolant 124 are arranged in the coolant arrangement 120 such that no direct contact is possible between the first and second coolants 122, 124, on the one hand, and the gas 105 being cooled, on the other hand. Such a direct contact is not possible in the exemplary embodiment being shown due to the fact that the first and second coolants 122, 124 are arranged each in a number of heat exchanger plates 130, which completely enclose the respective coolant. The gas 105 being cooled is now passed through between the heat exchanger plates 130. The heat exchanger plates 130 filled with the first coolant 122 form a respective first area 126 and the heat exchanger plates 130 filled with the second coolant 124 form a respective second area 128. The heat exchanger plates are arranged alternatingly in relation to one another such that the gas 105 being cooled is guided, while it is being passed through between two adjacent heat exchanger plates 130, through at least one first area 126 having the first coolant 122 and through at least one second area 128 having the second coolant 124. This makes possible an especially homogeneous temperature distribution of the gas 106 reaching the gas outlet 114.

The plurality of heat exchanger plates 130 have a plate housing 132 and a closure (not shown). The heat exchanger plates may be comprised of metal or plastic material portions forming the housing. A respective coolant is filled into the heat exchanger plates 130 in the unclosed state of the plate housing 132. The closure in the example being shown is a permanent closure unsuitable for temporary opening, which is embodied by a welding of the metal or plastic portions that make up the plate housing 132. In one exemplary embodiment, not shown, the closure is a detachable closure, for example, a screw cap. The heat exchanger plates 130 are inserted into the device housing 110 via a plate-mounting track (not shown) arranged at the device housing 110.

Either the first coolant 122 or the second coolant 124 is introduced into a particular heat exchanger plate 130. In one exemplary embodiment, not shown, at least one additional coolant is used in the cooling device according to the present invention. A mixture of at least two different coolants is introduced into a heat exchanger plate in another exemplary embodiment, not shown.

The first coolant 122 is a PCM coolant consisting of paraffin or a salt material, namely, PCM31 in this case. The second coolant 124 is a PCM coolant consisting of paraffin or a salt material, namely, PCM37 in this case. One coolant is water in one exemplary embodiment, not shown.

Thus, the first coolant 122 and the second coolant 124 have different melting points T1 and T2 according to the present invention. Thus, PCM31 has a melting point of 31° C. and PCM37 has a melting point of 37° C. The first melting point T1 and the second melting point T2 are each lower than 50° C., especially lower than 45° C. and preferably lower than 40° C. in other exemplary embodiments as well. It can be ensured by such low melting points that the inhalation gas inhaled by the user of the closed-circuit respirator 108 will not become unpleasantly hot.

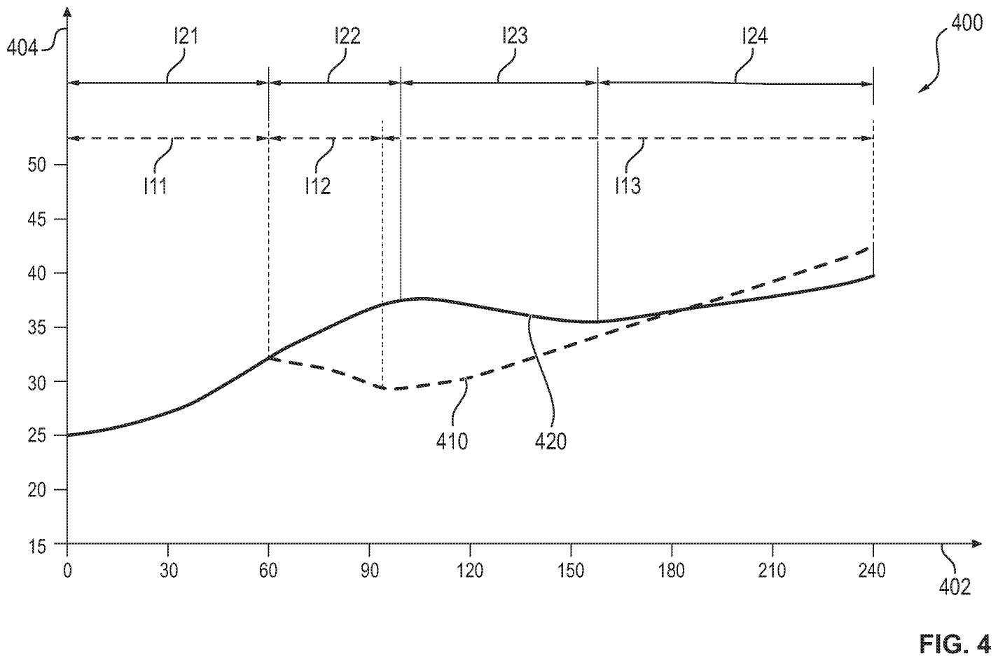

A key advantage of the use according to the present invention of a first coolant with a first melting point T1, which is different from the second melting point T2 of the second coolant, is that an absorption of heat takes place within the framework of the enthalpy of fusion of the corresponding coolant at different temperatures of the gas 105 being cooled and thus at different times during a use of the closed-circuit respirator 108. As a result, a temperature curve of the inhaled gas can be preset by selecting the compositions of different coolants. Such a setting is shown in detail in connection with FIG. 4.

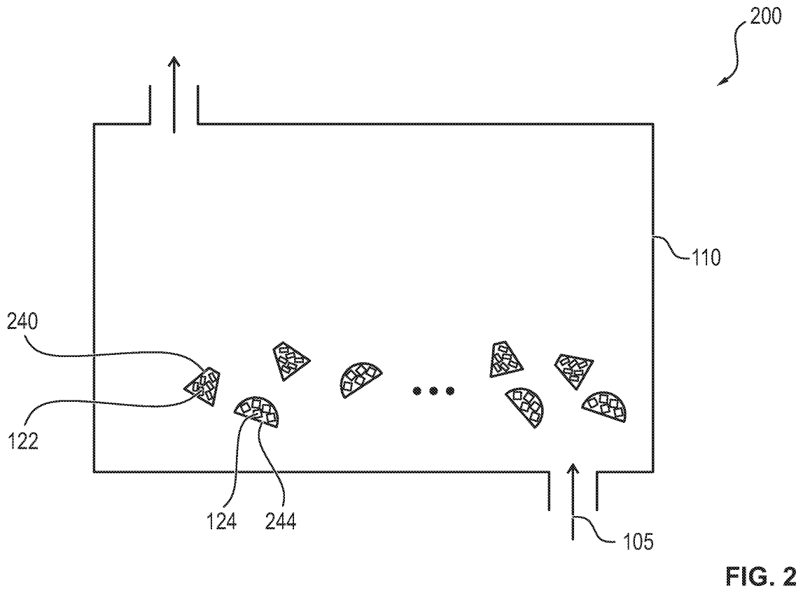

FIG. 2 shows a schematic view of a second exemplary embodiment of the cooling device 200 according to the present invention.

The cooling device 200 differs from the cooling device 100 shown in FIG. 1 only in that the first and second coolants 122, 124 are not provided in heat exchanger plates, but by a plurality of individually sealed capsules 240, 244 filled with a respective coolant. The air to be cooled can only be guided as a result through the existing layer of capsules 240, 244. The capsules 240 filled with the first coolant 122 have a frustoconical configuration. The capsules 244 filled with the second coolant 124 have a hemispherical configuration. Such a shaping, especially the use of different shapes, makes it possible to provide intermediate spaces, through which the air 105 to be cooled can be passed. In one exemplary embodiment, not shown, at least some of the capsules have a conical shape, a pyramid shape, a truncated pyramid shape or a tetrapod shape.

In the exemplary embodiment shown, the plurality of capsules 240, 244 are filled as a bulk material into the device housing 110. The capsules are comprised of metal or plastic material. In one exemplary embodiment, not shown, the device housing has a capsule rack, which is configured to accommodate a plurality of capsules. The capsule rack is preferably arranged in the device housing such that the gas to be cooled is guided between the gas inlet and the gas outlet along the capsule rack or is passed through the capsule rack.

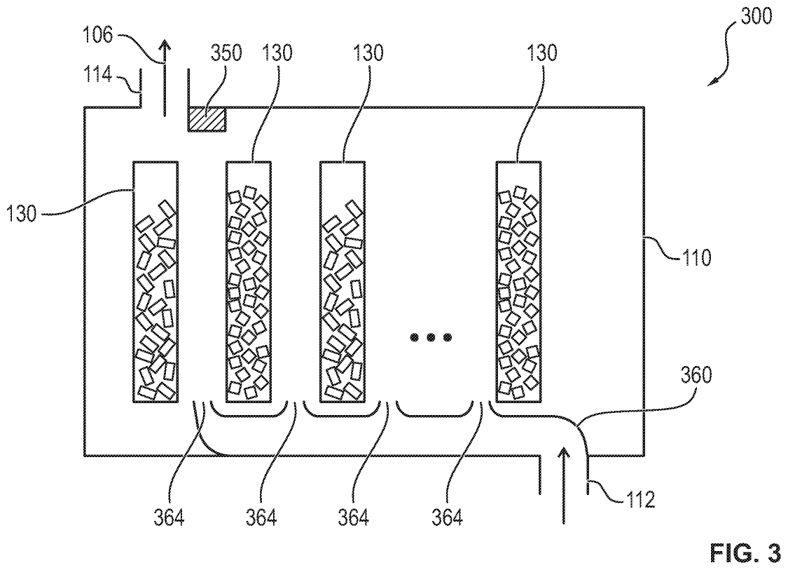

FIG. 3 shows a schematic view of a third exemplary embodiment of the cooling device 300 according to the present invention.

The cooling device 300 corresponds to the cooling device 100, and a temperature sensor (temperature sensor/RFID tag) 350 and a distribution device 360 are additionally arranged in the device housing 110.

The temperature sensor/RFID tag 350 is a temperature sensor unit embodied with a passive RFID (radio frequency identification) tag and one or more temperature sensing elements that determines a temperature within the device housing 110 by means of the one or more temperature sensing elements. The temperature sensor/RFID tag 350 is arranged in an area of the gas outlet 114. As a result, a temperature of the already cooled gas 106 can advantageously be determined at the gas outlet 114. The temperature sensor/RFID tag 350 is configured, furthermore, to output the determined temperature in the presence of a corresponding external temperature polling. Such an external temperature polling is outputted in this case by a temperature-reading unit, which is arranged in the closed-circuit respirator and which is embodied here by an RFID reading unit. The temperature-reading unit is not a part of the cooling device according to the present invention and is not shown in FIG. 3.

The distribution device 360 is arranged in the area of the gas inlet 112. It is configured to guide the gas 106 to be cooled from the gas inlet 112 in guide ducts formed by a plurality of heat exchanger plates 130. The distribution device 360 has for this purpose a plurality of gas outlet openings 364 corresponding to the existing plurality of heat exchanger plates 130. In an especially advantageous exemplary embodiment, not shown, the heat exchanger plates are spaced at differently spaced locations from one another corresponding to a gas pressure within the distribution device. As a result, an essentially homogeneous stream of the gas being cooled can be obtained within the device volume.

FIG. 4 shows a diagram 400, which shows a temperature curve 410, 420 of an inhaled gas over time for a cooling device according to the present invention.

The abscissa 402 of the diagram 400 describes a duration of use, the use of the respective closed-circuit respirator in minutes. The coordinate 404 of the diagram 400 describes the temperature of the inhaled gas provided by the closed-circuit respirator in degrees Celsius.

The temperature curve 410, drawn in broken line, shows the temperature of the inhaled gas provided over the duration of use for a cooling device not according to the present invention, which cools with a single coolant. The coolant is the PCM cooling material PCM31, which has a melting point of 31° C.

The temperature curve 410 is characterized by a first time interval I11, in which the temperature rises slowly beginning from a starting temperature at 25° C., so that the melting point of the coolant PCM31 is reached after about 60 minutes. The temperature then drops over about 30 minutes in a second time interval I12 before it slowly rises again within the final, third time interval I13 until the end of the use after 240 minutes. The lowering of the temperature in the second time interval I12 by about 4° C. can be explained by the enthalpy of fusion needed for melting the coolant. The enthalpy of fusion describes the quantity of energy that is needed to overcome the attracting intramolecular forces within the coolant during the transition from the solid state to the liquid state of the coolant. This energy is extracted from the gas being cooled, so that an additional cooling of this gas takes place in the range of the melting point.

The temperature curve 420 drawn in solid line shows the temperature of the provided inhaled gas over the duration of use for a cooling device according to the present invention, which cools with a first coolant and with a second coolant. The first coolant is again the PCM cooling material PCM 31, which has a melting point of 31° C. The second coolant is the PCM cooling material PCM37, which has a melting point of 37° C. The first coolant and the second coolant are provided at a ratio of 1:1 within the cooling device according to the present invention.

The temperature curve 420 does not differ from the temperature curve 410 in the first time interval I21. In the second time interval I22, which begins when the melting point of 31° C. of the first coolant is reached, there is only a reduction of the temperature increase compared to the first time interval I11, but there is no reduction of the temperature. A third time interval I23 begins after the melting point of 37° C. of the second coolant is reached, and a reduction of the temperature by about 3° C. can be seen. In a fourth time interval I24, the temperature of the inhaled gas rises again slowly until the end of the use after 240 minutes. The behavior of the temperature curve 420 after the melting point of the first coolant and after the melting point of the second coolant can again be understood as being due to the additional removal of energy from the gas being cooled based on the respective enthalpy of fusion.

The comparison between the temperature curve 410 and the temperature curve 420 according to the present invention shows that the temperature of the inhaled gas is permanently below the temperature of a cooling device not according to the present invention after about 180 minutes. Higher temperatures of the inhaled gas are accepted, in return, in the middle of the use. The device according to the present invention can therefore be more suitable for a longer duration of use at the currently selected coolant ratio, because a critical temperature of the inhaled gas, equaling about 42° C., is not exceeded for a longer duration of use.

Depending on a planned duration of use of the closed-circuit respirator and of an assumable outside temperature during the use, another coolant with a different melting point, or another ratio of the quantities of coolants may be especially advantageous. A ratio, for example, 1:2 or 2:1 would be able to be handled in practice. The use of capsules, as it is shown in FIG. 2, is especially advantageous for providing a certain ratio of the quantity of the first coolant to the quantity of the second coolant.

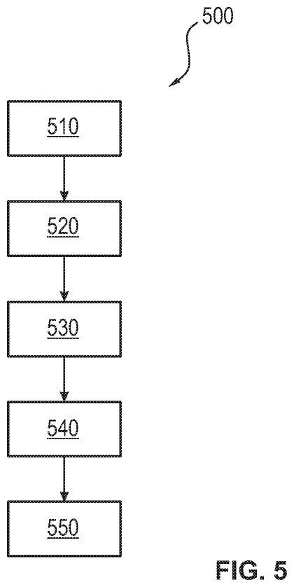

FIG. 5 shows a flow chart of an exemplary embodiment of the process 500 according to the other aspect of the present invention.

The process 500 for operating a closed-circuit respirator has the steps mentioned below.

A first step 510 comprises the provision of a device housing, which has a housing wall, which encloses a device volume.

A further step 520 comprises the provision of a first coolant with a first melting point T1 and of a second coolant with a second melting point T2 in the device volume, the first melting point T1 being different from the second melting point T2.

A next process step 530 comprises the admission of a gas to be cooled into the device housing.

A next step 540 of the process according to the present invention comprises a cooling of the gas to be cooled by the first coolant and by the second coolant, while no direct contact is possible between the gas being cooled and the first or second coolant.

A final step 550 comprises the letting out of the gas having passed through the device housing from the device housing.

The process steps 530, 540, 550 are carried out according to the present invention repeatedly within the closed-circuit respirator in order to ensure a lasting cooling of the gas being cooled within the breathing gas circuit.

The process step 510 is carried out once during the manufacture of the closed-circuit respirator. Step 520 is carried out typically depending on the use conditions to be expected prior to the use, the two coolants being provided in the frozen state.

In one exemplary embodiment, not shown, the cooling of the gas to be cooled according to step 540 has, furthermore, the steps of
guiding the gas being cooled through at least one first area of the first coolant, and
guiding the gas being cooled through at least one second area of the device volume containing the second coolant.

In an especially preferred exemplary embodiment of the process according to the present invention, which exemplary embodiment is not shown, the first and second coolants are provided according to step 520 at a predefined ratio. The predefined ratio is set, for example, by taking into consideration an expected duration of use and/or an expected outside temperature during a use of the closed-circuit respirator. The expected temperature curve of the inhaled gas over the duration of use is to be taken into consideration here analogously to the discussion in connection with FIG. 4 depending on the existing melting points.

All the exemplary embodiments discussed may also be configured according to the present invention for more than two different coolants. As a result, the assumable temperature curve can likewise be adapted to existing conditions of use corresponding to the existing melting points.

While specific embodiments of the invention have been shown and described in detail to illustrate the application of the principles of the invention, it will be understood that the invention may be embodied otherwise without departing from such principles.

### LIST OF REFERENCE NUMBERS

100, 200, 300 Cooling device

104 Breathing gas circuit

105 Gas to be cooled

106 Gas at the gas outlet

108 Closed-circuit respirator

110 Device housing

112 Gas inlet

114 Gas outlet

116 Device volume

118 Housing wall

120 Coolant arrangement

122 First coolant

124 Second coolant

126 First area

128 Second area

130 Heat exchanger plate

132 Plate housing

240 Capsule containing first coolant

244 Capsule containing second coolant

350 Temperature sensor cunit—Temperature sensor/RFID tag

360 Distribution device

364 Gas outlet opening

400 Diagram

402 Abscissa

404 Ordinate

410 Temperature curve (implementation not according to the present invention)

420 Temperature curve (implementation according to the present invention)

500 Process

510, 520, 530, 540, 550 Process steps

I11, I12, I13 Time intervals (implementation not according to the present invention)

I21, I22, I23, I24 Time intervals (implementation according to the present invention)

What is claimed is:

1. A cooling device for a closed-circuit respirator, the cooling device comprising:
a device housing comprising a housing wall defining a device volume, the device housing being configured to be connected to a breathing gas circuit of the closed-circuit respirator, the device housing having a gas inlet configured to admit gas of the breathing gas circuit to be cooled into the device housing, and having a gas outlet configured to let gas admitted into the device housing through the gas inlet out of the device housing, the device housing being configured such that the gas to be cooled flows from the gas inlet through the device volume to the gas outlet; and
a coolant arrangement comprising a first coolant with a first melting point and a second coolant with a second melting point, wherein: the coolant arrangement is arranged in the device volume; the first coolant and the second coolant are configured such that no direct contact is possible between the first coolant and the gas being cooled and no direct contact is possible between the second coolant and the gas being cooled; and the first melting point is different from the second melting point.

2. A cooling device in accordance with claim 1, wherein the first melting point and the second melting point are each lower than 50° C.

3. A cooling device in accordance with claim 1, wherein:
the cooling arrangement is configured such that the gas to be cooled is guided from the gas inlet through the coolant arrangement to the gas outlet; and
the gas to be cooled is guided by the configuration of the cooling arrangement to pass through at least one first area having the first coolant and through at least one second area having the second coolant.

4. A cooling device in accordance with claim 1, wherein the first coolant and/or the second coolant are formed by a phase-change material as a cooling material consisting essentially of paraffin or a salt material.

5. A cooling device in accordance with claim 1, wherein:
the configuration of the first coolant such that no direct contact is possible between the first coolant and the gas being cooled comprises a plurality of individually sealed capsules each filled with the first coolant; or
the configuration of the second coolant such that no direct contact is possible between the second coolant and the gas being cooled comprises a plurality of individually sealed capsules each filled with the second coolant; or
the configuration of the first coolant such that no direct contact is possible between the first coolant and the gas being cooled comprises a plurality of individually sealed capsules each filled with the first coolant and the configuration of the second coolant such that no direct contact is possible between the second coolant and the gas being cooled comprises a plurality of individually sealed capsules each filled with the second coolant.

6. A cooling device in accordance with claim 1, wherein:
the configuration of the first coolant such that no direct contact is possible between the first coolant and the gas being cooled comprises a plurality of sealed heat exchanger plates each filled with the first coolant; or
the configuration of the second coolant such that no direct contact is possible between the second coolant and the gas being cooled comprises a plurality of sealed heat exchanger plates each filled with the second coolant; or
the configuration of the first coolant such that no direct contact is possible between the first coolant and the gas being cooled comprises a plurality of sealed heat exchanger plates each filled with the first coolant and the configuration of the second coolant such that no direct contact is possible between the second coolant and the gas being cooled comprises a plurality of sealed heat exchanger plates each filled with the second coolant.

7. A cooling device in accordance with claim 6, wherein the coolant arrangement is formed by an alternating sequence of heat exchanger plates filled with the first coolant and of heat exchanger plates filled with the second coolant.

8. A cooling device in accordance with claim 1, further comprising a temperature sensor arranged at or in the device housing and configured to output a measured temperature of the device volume.

9. A cooling device in accordance with claim 8, wherein the temperature sensor comprises a radio frequency identification tag with one or more temperature sensing elements arranged at or in the device housing, the radio frequency identification tag being configured to output a temperature currently present in the device volume in the presence of a corresponding external temperature polling signal.

10. A closed-circuit respirator comprising a cooling device, the cooling device comprising:
a breathing gas circuit; and
a cooling device comprising a device housing comprising a housing wall defining a device volume, the device housing being connected to the breathing gas circuit, the device housing having a gas inlet configured to admit gas of the breathing gas circuit to be cooled into the device housing, and having a gas outlet configured to let gas admitted into the device housing through the gas inlet out of the device housing, the device housing being configured such that the gas to be cooled flows from the gas inlet through the device volume to the gas outlet and a coolant arrangement comprising a first coolant with a first melting point and a second coolant with a second melting point, wherein: the coolant arrangement is arranged in the device volume; the first coolant and the second coolant are configured such that no direct contact is possible between the first coolant and the gas being cooled and no direct contact is possible between the second coolant and the gas being cooled; and the first melting point is different from the second melting point.

11. A closed-circuit respirator in accordance with claim 10, wherein:
the configuration of the first coolant such that no direct contact is possible between the first coolant and the gas being cooled comprises a plurality of individually sealed capsules each filled with the first coolant; or
the configuration of the second coolant such that no direct contact is possible between the second coolant and the gas being cooled comprises a plurality of individually sealed capsules each filled with the second coolant; or
the configuration of the first coolant such that no direct contact is possible between the first coolant and the gas being cooled comprises a plurality of individually sealed capsules each filled with the first coolant and the configuration of the second coolant such that no direct contact is possible between the second coolant and the gas being cooled comprises a plurality of individually sealed capsules each filled with the second coolant.

12. A closed-circuit respirator in accordance with claim 10, wherein:
the configuration of the first coolant such that no direct contact is possible between the first coolant and the gas being cooled comprises a plurality of sealed heat exchanger plates each filled with the first coolant; or
the configuration of the second coolant such that no direct contact is possible between the second coolant and the gas being cooled comprises a plurality of sealed heat exchanger plates each filled with the second coolant; or
the configuration of the first coolant such that no direct contact is possible between the first coolant and the gas being cooled comprises a plurality of sealed heat exchanger plates each filled with the first coolant and the configuration of the second coolant such that no direct contact is possible between the second coolant and the gas being cooled comprises a plurality of sealed heat exchanger plates each filled with the second coolant.

13. A closed-circuit respirator in accordance with claim 1, further comprising a temperature sensor arranged at or in the device housing and configured to output a measured temperature of the device volume, wherein the temperature sensor comprises a radio frequency identification tag with one or more temperature sensing elements arranged at or in the device housing, the radio frequency identification tag being configured to output a temperature currently present in the device volume in the presence of a corresponding external temperature polling signal.

14. A process for operating a closed-circuit respirator, the process comprising the steps of:
providing a device housing, which has a housing wall enclosing a device volume;
providing a first coolant, with a first melting point, in the device volume;
providing a second coolant, with a second melting point, in the device volume, the first melting point being different from the second melting point;
admitting a gas to be cooled into the device housing;
cooling the admitted gas by the first coolant and by the second coolant, wherein no direct contact is possible between the gas being cooled and the first coolant and no direct contact is possible between the gas being cooled and the second coolant; and
allowing gas that has passed through the device housing out of the device housing.

15. A process in accordance with claim 14, wherein the first melting point and the second melting point are each lower than 50° C.

16. A process in accordance with claim 14, wherein the cooling of the admitted gas comprises the steps of:
guiding the admitted gas through at least one first area of the device volume having the first coolant; and
guiding the admitted gas through at least one second area of the device volume having the second coolant.

17. A process in accordance with claim 14, wherein the first coolant and the second coolant are provided at a predefined ratio and wherein the predefined ratio is set based on an expected duration of use of the closed-circuit respirator.

18. A process in accordance with claim 14, wherein the first coolant and the second coolant are provided at a predefined ratio, and wherein the predefined ratio is set based on an expected ambient temperature during the use of the closed-circuit respirator.

19. A process in accordance with claim 14, further comprising arranging a temperature sensor at or in the device housing and configured to output a measured temperature of the device volume.

20. A process in accordance with claim 19, wherein the temperature sensor comprises a radio frequency identification tag with one or more temperature sensing elements arranged at or in the device housing, the radio frequency identification tag being configured to output a temperature currently present in the device volume in the presence of a corresponding external temperature polling signal.
